(19) United States
(12) Patent Application Publication
(10) Pub. No.: US 2019/0117530 A1
(43) Pub. Date: Apr. 25, 2019

(54) NANOEMULSION COMPOSITIONS AND METHODS

(71) Applicant: Fernando Thome Kreutz, Porto Alegre (BR)

(72) Inventors: Fernando Thome Kreutz, Porto Alegre (BR); Alana Carina Klein, Porto Alegre (BR); Sarah Ledur Selbach, Porto Alegre (BR)

(21) Appl. No.: 15/766,287

(22) PCT Filed: Oct. 7, 2016
(86) PCT No.: PCT/BR16/50253

§ 371 Date: Apr. 5, 2018

Related U.S. Application Data
(60) Provisional application No. 62/239,385, filed on Oct. 9, 2015.

(51) Int. Cl. A61K 8/06; A61K 8/86; A61K 8/34; A61K 8/37; A61K 8/92; A61K 8/49; A61Q 13/00

(52) U.S. Cl. CPC A61K 8/062; A61K 8/86; A61K 8/34; A61K 8/37; A61K 8/922; A01N 25/04; A61Q 13/00; A61K 2800/21; A61K 2800/522; A61K 2800/524; A61K 8/49

(57) ABSTRACT

A composition comprises a fragrance and/or oil and a surfactant, wherein the composition is free of lower alkyl alcohols and wherein the composition is a nanoemulsion.

### FIELD

The present invention relates to nanoemulsions. In particular, the present invention relates to nanoemulsion compositions and methods of making these compositions.

### BACKGROUND

Fragrance compositions, such as room diffusers, are generally used to aromatize different spaces and/or objects. They are generally prepared using ethanol, which aids in vaporizing the aroma from the composition.

Nanotechnology has been used in many fields, such as electronics, engineering, and pharmaceuticals. U.S. Patent Application Publication Nos. 2013/0011454 and U.S. Pat. No. 7,476,393 are directed to cosmetic nanoemulsions that contain alcohol solvents.

U.S. Pat. No. 6,774,101 describes an alcohol-free translucent perfuming composition in the form of a low viscosity, vaporizable oil-in-water emulsion containing at least a perfuming ingredient, a surfactant system having a hydrophilic-lipophilic ratio not less than 10 and water. The exemplified compositions of U.S. Pat. No. 6,774,101 all use Steareth-20 and Steareth-21 in combination or Oleth-20 alone. Furthermore, all of the exemplified compositions of U.S. Pat. No. 6,774,101 use an organic solvent as a stabilizer, namely Isopar™ (a synthetic isoparaffin hydrocarbon solvent) or Gemseal™ (a C₁₈-C₂₁ alkane solvent).

International Patent Application Publication No. WO 2011/077062 describes alcohol-free perfuming compositions in the form of a nanodispersion including a continuous aqueous phase and a liquid dispersed oily phase, in which said aqueous phase includes water and at least one non-ionic stabilising agent and said oily phase includes at least one flavouring agent. The exemplified compositions of International Patent Application Publication No. WO 2011/077062 all use Massocare™ HCO 40 (PEG-40 hydrogenated castor oil), Lipocol™ HCO 60 (PEG-60 hydrogenated castor oil), Myrj™ S20, S50, or S100 (PEG-20, -50, or -100 stearate), and/or PEG-3 oleate.

European Patent Application No. 2719371 describes an aqueous alcohol-free cologne composition that comprises at least one glycol solvent selected from the group consisting of ethylene glycol, propylene glycol, ethoxydiglycol, polyethylene glycols and polyethylene glycol-conjugates, in an amount sufficient to solubilize the other components.

U.S. Pat. No. 5,468,725 describes an alcohol-free transparent perfume containing an alcohol-free perfume base, water, a stable transparent oil-in-water microemulsion flavor concentrate formed of water, at least one hydrophobic perfume oils, at least one cationic surfactant and at least one non-ionic surfactant in the absence of lower alkanols. U.S. Pat. No. 5,468,725 states that it is an essential feature that one or more cationic surfactants be present to enable the formation of a desired perfume formulation.

European Patent No. 2181690 describes perfume compositions with high perfume oil content and reduced or no alcohol content of C₂-C₅ monovalent alcohols, especially ethanol. In the oil phase, a solvent is present having a log P value of >5 and is selected from isododecane, isohexadecane, isoeicosane, isoparaffine fluids, C₁₃-C₃₀alkanes, hydrogenated didecene, hydrogenated didodecene, hydrogenated polydecene, hydrogenated polydodecene, hydrogenated tridodecene, hydrogenated polyisobutene, mineral oils, and a mixture of two or more thereof or the solvent is a linear silicone, a cyclic silicone or a mixture thereof. In the water phase, an emulsifier is present that is a mixture of a first non-ionic emulsifier, an anionic emulsifier and optionally a second non-ionic emulsifier.

U.S. Pat. No. 5,374,614 is directed to clear oil in water microemulsions comprising a perfume oil, an aqueous phase and one or more surfactants with a hydrophilic-lipophilic balance between 9 and 18, and co-surfactants of which at least 0.5% are ionic co-surfactants.

U.S. Patent Application Publication No. 2012/0097754 is directed to air freshener compositions that comprise an amphiphilic solubilizing agent that includes one or more anionic, cationic or amphoteric surfactants or a combination thereof.

U.S. Pat. No. 7,226,901 is directed to stable perfuming compositions in the form of transparent fluid water-in-oil or oil-in-water emulsions, obtained by addition of a volatile fluorinated oil to the oily phase, capable of bringing closer the densities of the respective dispersed and continuous phases.

U.S. Pat. No. 5,283,056 and Chinese Patent No. 103637942 are directed to further ethanol-free perfume compositions.

A need exists for the development of a product, composition and/or method that provides the public with a useful alternative.

### SUMMARY OF THE INVENTION

In accordance with an aspect, there is provided a composition comprising a fragrance and/or oil, a surfactant, and an aqueous medium, wherein the composition is free of lower alkyl alcohols and wherein the composition is a nanoemulsion.

In an aspect, the surfactant is non-ionic.

In an aspect, the surfactant is ethoxylated.

In an aspect, the surfactant comprises at least a first ethoxylated fatty alcohol of the formula:

CH₃CH₂ₘO—CH₂—CH₂ₙOH;

wherein m is from about 4 to about 40 and n is from about 1 to about 60.

In an aspect, m is from about 12 to about 20.

In an aspect, n is from about 1 to about 24.

In an aspect, the composition comprises a second ethoxylated fatty alcohol of the formula:

CH₃CH₂ₘO—CH₂—CH₂ₙOH;

wherein m is from about 4 to about 40 and n is from about 1 to about 60, wherein the second ethoxylated alcohol is different from the first ethoxylated alcohol.

In an aspect, m and n in the first ethoxylated alcohol are from about 12 to about 20 and from about 1 to about 10, respectively, and wherein m and n in the second ethoxylated alcohol are from about 12 to about 20 and from about 11 to about 30, respectively.

In an aspect, the surfactant comprises oleth-20, laureth-2, laureth-4, laureth-23, or a combination thereof.

In an aspect, the surfactant consists of oleth-20 and laureth-2, oleth-20 and laureth-4, laureth-23 and laureth-4, or laureth-23 and laureth-2.

In an aspect, the surfactant consists of oleth-20.

In an aspect, the surfactant is present in the composition in a total amount of from about 0.5% w/w to about 20% w/w, such as from about 0.5% w/w, about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, or about 19% w/w to about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, or about 20% w/w.

In an aspect, the surfactant is present in the composition in a total amount of from about 5% w/w to about 18% w/w or from about 6% w/w to about 17% w/w.

In an aspect, the fragrance is present in the composition in an amount of from about 0.1% to about 30% w/w, such as from about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, or about 29% to about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w.

In an aspect, the fragrance is present at about 10%.

In an aspect, the fragrance is present at about 5%.

In an aspect, the fragrance is present at about 2%.

In an aspect, the fragrance comprises an essential oil.

In an aspect, the essential oil is citronella oil or lemongrass oil.

In an aspect, the oil is andiroba oil.

In an aspect, the composition further comprises a preservative.

In an aspect, the preservative is selected from antimicrobial agents, small carboxylic acids and salts thereof, formaldehyde-releasers, isothiazolinones, phenoxyethanol, and combinations thereof.

In an aspect, the preservative is an isothiazolinone, such as a mixture of methylchloroisothiazolinone and methylisothiazolinone, preferably in a 3:1 ratio.

In an aspect, the preservative is present in an amount of from about 0.01% to about 1% w/w, such as from about 0.01%, about 0.05%, about 0.1%, about 0.15%, about 0.2%, about 0.25%, about 0.5%, or about 0.75% to about 0.05%, about 0.1%, about 0.15%, about 0.2%, about 0.25%, about 0.5%, about 0.75%, or about 1% w/w, such as about 0.15% w/w.

In an aspect, the composition further comprises an antioxidant.

In an aspect, the antioxidant is selected from ascorbic acid, erythorbic acid, a botanical extract, such as rosemary extract, green tea extract, or other extract containing a polyphenol antioxidant, vitamin E, tocopherols, ascorbyl palmitate, butylated hydroxyanixole (BHA), butylated hydroxytoluene (BHT), pentaerythrityl tetra-di-t-butyl hydroxyhydrocinnamate, and combinations of any thereof.

In an aspect, the antioxidant is BHT.

In an aspect, the antioxidant is pentaerythrityl tetra-di-t-butyl hydroxyhydrocinnamate.

In an aspect, the nanoemulsion has an average droplet size of from about 0.1 nm to about 1000 nm, such as from about 0.1 nm, about 1 nm, about 10 nm, about 20 nm, about 30 nm, about 40 nm, about 50 nm, about 100 nm, about 200 nm, about 300 nm, about 400 nm, about 500 nm, about 600 nm, about 700 nm, about 800 nm, or about 900 nm to about 1 nm, about 10 nm, about 20 nm, about 30 nm, about 40 nm, about 50 nm, about 100 nm, about 200 nm, about 300 nm, about 400 nm, about 500 nm, about 600 nm, about 700 nm, about 800 nm, about 900 nm, or about 1000 nm.

In an aspect, the nanoemulsion has an average droplet size of from about 1 nm to about 100 nm, from about 20 nm to about 50 nm, or from about 10 nm to about 30 nm.

In an aspect, the aqueous medium is water.

In an aspect, the composition is non-turbid.

In an aspect, the composition is stable.

In an aspect, the composition is free of an oleth-based surfactant.

In an aspect, the composition is free of oleth-20.

In an aspect, the composition is free of a steareth-based surfactant.

In an aspect, the composition is free of steareth-20 and/or steareth-21.

In an aspect, the composition is free of glycols.

In an aspect, the composition is free of an ionic surfactant.

In an aspect, the composition is free of ester surfactants.

In an aspect, the composition is free of ester ether surfactants.

In an aspect, the composition is free of pegylated fatty acids.

In an aspect, the composition is free of fatty acids.

In accordance with another aspect, there is provided a method of making a composition, the method comprising:

heating a mixture of a fragrance and/or oil and a surfactant until it reaches a cloud point temperature; and

adding an aqueous medium to rapidly cool the mixture, wherein the composition is a nanoemulsion.

In an aspect, the aqueous medium is added in a first portion and a second portion and wherein heating the mixture of the fragrance and/or oil and the surfactant until it reaches a cloud point comprises:

heating the mixture of the fragrance and/or oil and the surfactant to a first temperature;

adding the first portion of the aqueous medium to the mixture; and

heating the mixture until it reaches the cloud point temperature.

In an aspect, the first portion of the aqueous medium is from about 20% to about 80% of the total amount of the aqueous medium, such as from about 20%, about 25%, about 30%, about 35%, about 40%, about 45%, about 50%, about 55%, about 60%, about 65%, about 70%, or about 75% to about 25%, about 30%, about 35%, about 40%, about 45%, about 50%, about 55%, about 60%, about 65%, about 70%, about 75%, or about 80% of the total amount of the aqueous medium.

In an aspect, the first portion of the aqueous medium is about 40% of the total amount of the aqueous medium.

In an aspect, the first temperature is from about 40° C. to about 80° C., such as from about 40° C., about 45° C., about 50° C., about 55° C., about 60° C., about 65° C., about 70° C., or about 75° C., to about 45° C., about 50° C., about 55° C., about 60° C., about 65° C., about 70° C., about 75° C., or about 80° C.

In an aspect, the first temperature is about 70° C.

In an aspect, the cloud point temperature is from about 85° C. to about 100° C., such as from about 85° C., about 86° C., about 87° C., about 88° C., about 89° C., about 90° C., about 91° C., about 92° C., about 93° C., about 94° C., about 95° C., about 96° C., about 97° C., about 98° C., or about 99° C. to about 86° C., about 87° C., about 88° C., about 89° C., about 90° C., about 91° C., about 92° C., about 93° C., about 94° C., about 95° C., about 96° C., about 97° C., about 98° C., about 99° C., or about 100° C.

In an aspect, the cloud point temperature is about 95° C.

In an aspect, the cloud point temperature is maintained for from about 1 to about 15 minutes, such as from about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, or about 14 minutes to about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes.

In an aspect, the cloud point temperature is maintained for about 5 minutes.

In an aspect, the mixture is cooled to a temperature of from about 25° C. to about 60° C., such as from about 25° C., about 30° C., about 35° C., about 40° C., about 45° C., about 50° C., or about 55° C. to about 30° C., about 35° C., about 40° C., about 45° C., about 50° C., about 55° C., or about 60° C.

In an aspect, the composition is cooled to a temperature of about 40° C.

In an aspect, the components are stirred during heating and/or cooling.

In an aspect, the surfactant is non-ionic.

In an aspect, the surfactant is ethoxylated.

In an aspect, the surfactant comprises at least a first ethoxylated fatty alcohol of the formula:

CH₃CH₂ₘO—CH₂—CH₂ₙOH;

wherein m is from about 4 to about 40 and n is from about 1 to about 60.

In an aspect, m is from about 12 to about 20.

In an aspect, n is from about 1 to about 24.

In an aspect, the composition comprises a second ethoxylated fatty alcohol of the formula:

CH₃CH₂ₘO—CH₂—CH₂ₙOH;

wherein m is from about 4 to about 40 and n is from about 1 to about 60, wherein the second ethoxylated alcohol is different from the first ethoxylated alcohol.

In an aspect, m and n in the first ethoxylated alcohol are from about 12 to about 20 and from about 1 to about 10, respectively, and wherein m and n in the second ethoxylated alcohol are from about 12 to about 20 and from about 11 to about 30, respectively.

In an aspect, the surfactant comprises oleth-20, laureth-2, laureth-4, laureth-23, or a combination thereof.

In an aspect, the surfactant consists of oleth-20 and laureth-2, oleth-20 and laureth-4, laureth-23 and laureth-4, or laureth-23 and laureth-2.

In an aspect, the surfactant consists of oleth-20.

In an aspect, the surfactant is present in the composition in a total amount of from about 0.5% w/w to about 20% w/w, such as from about 0.5% w/w, about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, or about 19% w/w to about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, or about 20% w/w.

In an aspect, the surfactant is present in the composition in a total amount of from about 5% w/w to about 18% w/w or from about 6% w/w to about 17% w/w.

In an aspect, the fragrance is present in the composition in an amount of from about 0.1% to about 30% w/w, such as from about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, or about 29% to about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w.

In an aspect, the fragrance is present at about 10%.

In an aspect, the fragrance is present at about 5%.

In an aspect, the fragrance is present at about 2%.

In an aspect, the fragrance comprises an essential oil.

In an aspect, the essential oil is citronella oil or lemongrass oil.

In an aspect, the oil is andiroba oil.

In an aspect, the method further comprises adding a preservative once the mixture has cooled.

In an aspect, the preservative is selected from antimicrobial agents, small carboxylic acids and salts thereof, formaldehyde-releasers, isothiazolinones, phenoxyethanol, and combinations thereof.

In an aspect, the preservative is an isothiazolinone, such as a mixture of methylchloroisothiazolinone and methylisothiazolinone, preferably in a 3:1 ratio.

In an aspect, the preservative is present in an amount of from about 0.01% to about 1% w/w, such as from about 0.01%, about 0.05%, about 0.1%, about 0.15%, about 0.2%, about 0.25%, about 0.5%, or about 0.75% to about 0.05%, about 0.1%, about 0.15%, about 0.2%, about 0.25%, about 0.5%, about 0.75%, or about 1% w/w, such as about 0.15% w/w.

In an aspect, the method further comprises adding an antioxidant prior to heating the mixture.

In an aspect, the antioxidant is selected from ascorbic acid, erythorbic acid, a botanical extract, such as rosemary extract, green tea extract, or other extract containing a polyphenol antioxidant, vitamin E, tocopherols, ascorbyl palmitate, butylated hydroxyanixole (BHA), butylated hydroxytoluene (BHT), pentaerythrityl tetra-di-t-butyl hydroxyhydrocinnamate, and combinations of any thereof.

In an aspect, the antioxidant is BHT.

In an aspect, the antioxidant is pentaerythrityl tetra-di-t-butyl hydroxyhydrocinnamate.

In an aspect, the nanoemulsion has an average droplet size of from about 0.1 nm to about 1000 nm, such as from about 0.1 nm, about 1 nm, about 10 nm, about 20 nm, about 30 nm, about 40 nm, about 50 nm, about 100 nm, about 200 nm, about 300 nm, about 400 nm, about 500 nm, about 600 nm, about 700 nm, about 800 nm, or about 900 nm to about 1 nm, about 10 nm, about 20 nm, about 30 nm, about 40 nm, about 50 nm, about 100 nm, about 200 nm, about 300 nm, about 400 nm, about 500 nm, about 600 nm, about 700 nm, about 800 nm, about 900 nm, or about 1000 nm.

In an aspect, the nanoemulsion has an average droplet size of from about 1 nm to about 100 nm, from about 20 nm to about 50 nm, or from about 10 nm to about 30 nm.

In an aspect, the aqueous medium is water.

In an aspect, the composition is non-turbid.

In an aspect, the composition is stable.

In an aspect, the composition is free of an oleth-based surfactant.

In an aspect, the composition is free of oleth-20.

In an aspect, the composition is free of a steareth-based surfactant.

In an aspect, the composition is free of steareth-20 and/or steareth-21.

In an aspect, the composition is free of glycols.

In an aspect, the composition is free of an ionic surfactant.

In an aspect, the composition is free of ester surfactants.

In an aspect, the composition is free of ester ether surfactants.

In an aspect, the composition is free of pegylated fatty acids.

In an aspect, the composition is free of fatty acids.

In accordance with another aspect, there is provided a composition made by a method described herein.

In accordance with another aspect, there is provided a room diffuser comprising a composition described herein.

In accordance with another aspect, there is provided a perfume comprising a composition described herein.

In accordance with another aspect, there is provided an air freshener comprising a composition described herein.

In accordance with another aspect, there is provided an insect repellent comprising a composition described herein.

In accordance with another aspect, there is provided a cosmetic product comprising a composition described herein.

In an aspect, the cosmetic product is a moisturizer.

In accordance with another aspect, there is provided a use of a composition described herein as a base product for further formulation.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows the average droplet size (Zeta-average; d nm) and polydispersity (Pdl) of a fragrance composition, as measured by the equipment Zetasizer ZEN3600 (Malvern). This figure shows that the composition contains droplets in the nanometer size.

FIG. 2 shows the average droplet size (Zeta-average; d nm) and polydispersity (Pdl) of a fragrance composition, as measured by the equipment Zetasizer ZEN3600 (Malvern). This figure shows that the composition contains droplets in the nanometer size.

FIG. 3 shows the average droplet size (Zeta-average; d nm) and polydispersity (Pdl) of a nanoemulsion composition, as measured by the equipment Zetasizer ZEN3600 (Malvern). This figure shows that the composition contains droplets in the nanometer size.

FIG. 4 shows the average droplet size (Zeta-average; d nm) and polydispersity (Pdl) of a fragrance composition, as measured by the equipment Zetasizer ZEN3600 (Malvern). This figure shows that the composition contains droplets in the nanometer size.

FIG. 5 shows the olfactory characteristics of a fragrance composition comprising an essential oil and a vehicle, as measured using an olfactometer. The olfactometer uses gas chromatography analysis to quantify specific smells together with a trained technique to identify them.

FIG. 6 shows the olfactory characteristics of a fragrance composition comprising an essential oil in a nanoemulsion, as measured using an olfactometer. The olfactometer uses gas chromatography analysis to quantify specific smells together with a trained technique to identify them.

### DETAILED DESCRIPTION OF CERTAIN ASPECTS

Described herein are nanoemulsion compositions, as well as methods of making nanoemulsion compositions and related uses.

### Nanoemulsion Compositions

The following definitions are used herein and should be referred to for interpretation of the claims and the specification:

As used herein, the term “fragrance” means any odoriferous material. In general, such materials are characterized by a vapour pressure less than the atmospheric pressure at room temperatures. The fragrances employed herein will most often be liquid at room temperatures, but also can be solid such as the various camphoraceous fragrances known in the art. A wide variety of chemicals are known for fragrance uses, including materials such as aldehydes, ketones, esters, alcohols, terpenes and the like. Naturally occurring plant and animal oils and exudates comprising complex mixtures of various chemical components are known for use as fragrances, and such materials can be used herein. The fragrances herein can be relatively simple in their composition or can comprise highly sophisticated, complex mixtures of natural and synthetic chemical components, all chosen to provide any desired odour.

Typical fragrances that can be used in the compositions and methods described herein comprise, for example, woody/earthy bases containing exotic materials such as sandalwood oil, civet, patchouli oil, and the like. Other suitable fragrances are for example light, floral fragrances, for example, rose extract, violet extract, and the like. Fragrances can be formulated to provide desirable fruity odours, for example, lime, lemon, orange, and the like.

Thus, the term fragrance includes natural products such as essential oils. As used herein, the term “essential oils” means any concentrated hydrophobic liquid containing volatile aromatic compounds from plants.

Essential oils are typically highly scented droplets found in minute quantities in the flowers, fruit, stems, leaves, roots, wood, resin, and bark of aromatic plants. Typically, essential oils are highly fluid and exceptionally volatile.

Essential oils are typically complex mixtures of different organic molecules and may comprise, for example, terpenes, alcohols, esters, aldehydes, ketones, and/or phenols.

Synthetic fragrance oils are usually made from one or more of the constituents predominant within a particular essential oil; menthol, for example, often substitutes for mint and eucalyptol for eucalyptus.

Essential oils are typically recognized by their aromas and their compositions are typically determined through gas liquid chromatography (GLC). Essential oils can also have characteristic colours: eucalyptus is colourless; chamomile varies from white to blue; and others, such as basil and sandalwood (both light greenish-yellow), are in pastel shades. Yet others are richly pigmented, such as jasmine, which is a deep reddish-brown, patchouli, which is brown, and rose, which is orange-red.

Examples of essential oils include, but are not limited to, those extracted from anise, orange, basil, bergamot, birch, cajeput, chamomile (e.g., chamomile german, chamomile maroc, and chamomile roman), cinnamon (e.g., cinnamon zeylanicum), cedarwood, cyprus, citronella, clove buds, eucalyptus (e.g., eucalyptus globulus), fennel seeds, clove leaves, geranium, juniper berry, incense, lavender, hybrid lavender, frankincense, fennel, hyssop, juniper, lemongrass, lemon, marjoram, malaleuca alternifolia, tangerine, melissa, mint, myrrh, oregano, patchouli, scotch pine, mugo pine, grapefruit, mountain savoury, niaouli, red thyme, rose, rosemary, rose geranium, sage, sandalwood, thyme, tagestes, ginger, ylang ylang, or combinations thereof.

In short, any chemically compatible material that emanates a pleasant or otherwise desirable odour can be used as a fragrance in the compositions and methods described herein. Particular oils may be chosen due to specific effects, such as insect repellant effects in the case of citronella, lemongrass, and/or andiroba, for example.

Fragrance materials are described more fully in S. Arctander, Fragrance Flavors and Chemicals. Vols. I and II. Author, Montclair, N.J., and the Merck Index, 8^{th} Edition, Merck & Co., Inc. Rahway, N.J., both of which are incorporated herein by reference in their entirety.

The nanoemulsion compositions typically contain the fragrance component in an amount of from about 0.1% to about 30% w/w, such as from about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, or about 29% to about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w.

The compositions described herein may contain an oil, fat, wax, or similar product in addition to or instead of a fragrance essential oil. For example, certain oils may be used to hydrate skin and/or modify the consistency of the nanoemulsion. Particular examples of oils include, but are not limited to Carapa guianensis/andiroba oil, coconut oil, olive oil, palm oil, palm kernel oil, sunflower seed oil, safflower oil, hemp seed oil, corn oil, macadamia seed oil, green coffee oil, kukui nut oil, jojoba seed oil, sweet almond oil, avocado oil, castor seed oil, sulfated castor oil, argan nut oil, acai berry oil, andiroba nut oil, apricot kernel oil, soybean oil, baobab seed oil, black raspberry seed oil, blackberry seed oil, blackcurrant fruit oil, blueberry seed oil, borage seed oil, broccoli seed oil, marula kernel oil, cucumber seed oil, manketti oil, passion flower seed oil, camelina seed oil, linseed seed oil, strawberry seed oil, poppy seed oil, moringa oil, rice bran oil, pomegranate oil, pumpkin seed oil, walnut seed oil, fish oil, fish liver oil, cod liver oil, shark liver oil, vegetable oil, canola oil, peanut oil, sesame oil, flaxseed oil, grape seed oil, almond oil, cottonseed oil, groundnut oil, teaseed oil, walnut oil, cashew oil, colza oil, hazelnut oil, marula oil, mongongo nut oil, pecan oil, perilla oil, pine nut oil, pistachio oil, rapeseed oil, watermelon seed oil, diacylglycerol oil, and any combination thereof.

The nanoemulsion compositions typically contain the oil component in an amount of from about 0.1% to about 30% w/w, such as from about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, or about 29% to about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w.

As used herein, the term “emulsion” means a stable mixture of two immiscible substances in which one substance, the dispersed phase, is dispersed as tiny droplets within the other substance, the continuous phase. Typically, the emulsions described herein are oil-in-water nanoemulsions.

As used herein, the term “nanoemulsion” denotes an emulsion, typically an oil-in-water emulsion, having an average droplet size in the nanometer range. In some aspects, the nanoemulsions described herein containing a population of droplets having maximum and minimum diameters, wherein the difference between the maximum and minimum diameters does not exceed about 600 nm, about 550 nm, about 500 nm, about 450 nm, about 400 nm, about 350 nm, about 300 nm, about 250 nm, about 200 nm, about 150 nm, about 100 nm, about 90 nm, about 80 nm, about 70 nm, about 60 nm, about 50 nm, about 40 nm, about 30 nm, about 25 nm, about 20 nm, about 15 nm, about 10 nm, about 9 nm, about 8 nm, about 7 nm, about 6 nm, about 5 nm, about 4 nm, about 3 nm, about 2 nm, about 1 nm, or fewer nm.

In some aspects, droplets (e.g., fragrance- and/or oil-containing droplets) within the nanoemulsions described herein have diameters that are smaller than about 600 nm, about 550 nm, about 500 nm, about 450 nm, about 400 nm, about 350 nm, about 300 nm, about 250 nm, about 200 nm, about 150 nm, about 130 nm, about 120 nm, about 115 nm, about 110 nm, about 100 nm, about 90 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 15 nm, about 10 nm, about 5 nm, or fewer nm. Typically, the droplets have average diameters that are smaller than about 30 nm.

In some aspects, droplets (e.g., fragrance- and/or oil-containing droplets) within nanoemulsions described herein have diameters within the range of from about 0.1 nm to about 1000 nm, such as from about 0.1 nm, about 1 nm, about 10 nm, about 20 nm, about 30 nm, about 40 nm, about 50 nm, about 100 nm, about 200 nm, about 300 nm, about 400 nm, about 500 nm, about 600 nm, about 700 nm, about 800 nm, or about 900 nm to about 1 nm, about 10 nm, about 20 nm, about 30 nm, about 40 nm, about 50 nm, about 100 nm, about 200 nm, about 300 nm, about 400 nm, about 500 nm, about 600 nm, about 700 nm, about 800 nm, about 900 nm, or about 1000 nm. For example, the diameter may be from about 1 nm to about 100 nm, from about 20 nm to about 50 nm, from about 10 nm to about 30 nm, or from about 15 nm to about 30 nm.

In some aspects, a majority of the droplets (e.g., fragrance- and/or oil-containing droplets) within the nanoemulsions described herein have diameters below a specified size or within a specified range. In some embodiments, the majority is more than about 50%, about 60%, about 70%, about 75%, about 80%, about 85%, about 90%, about 95%, about 96%, about 97%, about 98%, about 99%, about 99.5%, about 99.6%, about 99.7%, about 99.8%, about 99.9% or more of the droplets in the nanoemulsion.

In some aspects, the nanoemulsions described herein are substantially free of droplets (e.g., fragrance- and/or oil-containing droplets) having diameters greater than about 120 nm, such as about 110 nm, about 100 nm, about 90 nm, about 80 nm, about 70 nm, about 60 nm, about 50 nm, about 40 nm, or about 30 nm.

In some aspects, the nanoemulsions described herein are stable. The term “stable” means the composition does not undergo any significant changes in average droplet size (Zeta-average), polydispersity and/ororganoleptic characteristics, such as smell, pH and/or appearance, during a given period of time at two different temperatures. For example, in a stable composition, the average droplet size may vary by about ±100%, by about ±90%, by about ±80%, by about ±70%, by about ±60%, by about ±50%, by about ±40%, by about ±30%, by about ±25%, by about ±20%, by about ±15%, by about ±10%, by about ±9%, by about ±8%, by about ±7%, by about ±6%, by about ±5%, by about ±4%, by about ±3%, by about ±2%, or by about ±1%, or less.

In some aspects, the fragrance and/or oil is present partially or entirely within nanodroplets of the nanoemulsions described herein; in some aspects, the fragrance and/or oil is adsorbed on the surface of nanodroplets of the nanoemulsions described herein; in some aspects, the fragrance and/or oil is associated with the interface between the nanodroplets and the dispersion medium. In some aspects, the fragrance and/or oil is found in two or more of these locations within the nanoemulsions described herein.

The term “surfactant” is short for surface active agent. Surfactants are amphiphilic compounds, meaning they contain two or more groups that, in their pure form, are insoluble in each other. Surfactants typically have at least one hydrophobic tail and at least one hydrophilic head and, more typically, surfactants have a single hydrophobic tail and a single hydrophilic head. Surfactants typically act to lower surface tension and can provide wetting, emulsification, foam, and detergency. It will be understood that any surfactant or combination of surfactants can be used in the nanoemulsion compositions described here, provided that the surfactant(s) and fragrance can together form a nanoemulsion. Thus, the surfactants described herein can be zwitterionic, amphiphilic, cationic, anionic, non-ionic, or combinations thereof and can include two or more surfactants from one such group or from different groups. Typically, the surfactants are non-ionic, as non-ionic surfactants tend to have low sensitivity to electrolytes, particularly divalent cations, as compared to ionic surfactants, and can be used with high salinity or hard water. In addition, many non-ionic surfactants are already in use in household or cosmetic products and are well-characterized and known to be safe for the purposes and in the quantities described herein.

In aspects, the non-ionic surfactant is an ethoxylated linear alcohol, an ethoxylated alkyl phenol, fatty acid esters, an amine or an amide derivative, an alkyl polyglucoside, an ethyleneoxide/propyleneoxide copolymers, a polyalcohol or ethoxylated polyalcohol, or a thiol (mercaptans) or a derivative. In a particular aspect, the non-ionic surfactant is an ethoxylated linear alcohol. Ethoxylated linear alcohols are typically named under the International Nomenclature of Cosmetic Ingredients (INCI) system, based on the hydrocarbon from which they are derived followed by their ethylene oxide number (EON), which represents the average number of ethylene oxide groups polycondensated to the hydrocarbon tail. For example, steareth-20 is derived from stearyl alcohol and each molecule has been polyethoxylated an average of 20 times. Ceteareth-20 comprises a mixture of polyoxyethylene ethers of cetyl alcohol and stearyl alcohol, which have an average number of 20 ethylene oxide residues in the polyoxyethylene chain. Laureth-2 is derived from lauryl alcohol and contains an average number of two ethylene oxide residues in the polyoxyethylene chain.

It will be understood that the polyethoxylated surfactant may have an EON of any number, such as from about 1 to about 60, such as from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60.

In certain aspects, when more than one polyethoxylated surfactant is used in the methods and compositions described herein, the polyethoxylated surfactants may have the same EON or may be selected so that their EON differs by at least one value. For example, the EON of the first polyethoxylated surfactant may be 1, 2, 3, 4, 5, 6, 7, 8, 9, or 10, whereas the EON of the second polyethoxylated surfactant may be 11, 12, 13, 14, 15, 16, 17, 18, 19, 20, 21, 22, 23, 24, 25, 26, 27, 28, 29, or 30. In another aspect, the EON of the two surfactants differs by a value of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more.

In aspects, the polyethoxylated surfactant may be saturated or unsaturated and branched or linear. Typically, the polyethoxylated surfactant is saturated and linear. The polyethoxylated surfactant may have a hydrocarbon tail length of from 5 carbons to about 40 carbons and is typically from about 12 to about 24 carbons. In certain aspects, when more than one polyethoxylated surfactant is used in the methods and compositions described herein, the polythoxylated surfactants may have the same length hydrocarbon tail or may be selected so that their hydrocarbon tail length differs by at least one value. For example, the hydrocarbon tail length of the polyethoxylated surfactants may be independently selected from 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more. In another aspect, the hydrocarbon tail length of the two surfactants differs by a value of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Typically, the surfactant is an ethoxylated fatty alcohol having a hydrocarbon tail comprising from about 5 to about 40 or more carbon atoms, as described above. Furthermore, when more than one surfactant is used, they may be selected to have the same or different hydrocarbon tail lengths. Likewise, the surfactant may have an EON of from about 1 to about 60, as described above and, when more than one surfactant is used, they may be selected to have the same or different EON. For example, it is contemplated to use two surfactants with the same tail lengths but different EONs; with different tail lengths but the same EONs; or with different tail lengths and different EONs. In another aspect, it is contemplated to use two surfactants with similar tail lengths and different EONs or with different tail lengths and similar EONs. It will be understood that the surfactants themselves may comprise a mixture of ethoxylated fatty alcohols, with the hydrocarbon tail length and EON specified being an average value. For example, ceteareth-20 comprises a mixture of high molecular weight ethoxylated fatty alcohols, mainly cetyl alcohol (hydrocarbon tail length of 16 carbon atoms) and stearyl alcohol (hydrocarbon tail length of 18 carbon atoms), which have been ethoxylated an average of 20 times.

In aspects, the ethoxylated fatty alcohols described herein may be referred to by the following general formula:

CH₃CH₂ₘO—CH₂—CH₂ₙOH;

Where in m is representative of the hydrocarbon tail length and is from about 4 to about 40 and n is representative of the EON and is from about 1 to about 60.

The compositions described herein comprise one or more surfactants in a total amount of from about 0.5% w/w to about 20% w/w, such as from about 0.5% w/w, about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, or about 19% w/w to about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, or about 20% w/w. Typically, the surfactants are used in a total amount of from about 4% w/w to about 10% w/w or from about 5% w/w to about 8% w/w.

When two or more surfactants are used in combination in the compositions and methods described herein, they may be used in various ratios. For example, when two surfactants are used, they may be used in a ratio of for example, from about 0.01:0.99 to about 0.99:0.01 (w/w), such as from about 0.10:0.90 to about 0.90:0.10 (w/w); from about 0.20:0.80 to about 0.80:0.20 (w/w); from about 0.3:0.7 to about 0.7:0.3 (w/w); or from about 0.6:0.4 to about 0.4:0.6 (w/w) based on the total mass of surfactant added.

The “hydrophilic-lipophilic balance (HLB) value” of a surfactant is a measure of the degree to which the surfactant is hydrophilic or lipophilic, determined by calculating values for the different regions of the molecule. The HLB value can be used to predict the surfactant properties of a molecule. A surfactant with an HLB value of less than 10 is generally lipid-soluble (water-insoluble), whereas a surfactant with an HLB value of greater than 10 is generally water-soluble (lipid-insoluble). A surfactant with an HLB value of from about 1.5 to about 3 is generally considered an anti-foaming agent. A surfactant with an HLB value of from about 3 to about 6 is generally considered a water in oil emulsifier. A surfactant with an HLB value of from about 7 to about 9 is generally considered a wetting and spreading agent. A surfactant with an HLB value of from about 13 to about 15 is generally considered a detergent. A surfactant with an HLB value of from about 12 to about 16 is generally considered an oil in water emulsifier. A surfactant with an HLB value of from about 15 to about 18 is generally considered a solubiliser or hydrotrope. More information on HLB values can be found in Griffin, W C. (1949), Journal of the Society of Cosmetic Chemists 1 (5): 311-26; Griffin, W C. (1954), Journal of the Society of Cosmetic Chemists 5 (4): 249-56; and Davies JT (1957), Gas/Liquid and Liquid/Liquid Interface (Proceedings of the International Congress of Surface Activity): 426-38, each of which is incorporated herein by reference in its entirety.

In an aspect, the surfactant(s) used in the compositions and methods described herein are specifically chosen so that the surfactant system has an HLB value of from about 8 to about 16, such as from about 8, about 9, about 10, about 11, about 12, about 13, about 14, or about 15 to about 9, about 10, about 11, about 12, about 13, about 14, about 15, or about 16. In an aspect, if two surfactants are used in the compositions and methods described herein, one such surfactant is chosen to have an HLB in the range of from about 3 to about 9, such as from about 3, about 4, about 5, about 6, about 7, or about 8 to about 4, about 5, about 6, about 7, about 8, or about 9, and the other surfactant is chosen to have an HLB in the range of from about 12 to about 18, such as from about 12, about 13, about 14, about 15, about 16, or about 17 to about 13, about 14, about 15, about 16, about 17, or about 18. In such a situation, the relative amounts of the surfactants are then chosen to arrive at an HLB value of from about 8 to about 16, as described above. It will be understood that any number of surfactants may be used in any amounts or ratios in the compositions described herein, with their various HLB values contributing to form a surfactant system with an HLB value of from about 8 to about 16.

In another aspect, the surfactant(s) chosen for use in the compositions and methods described herein are selected to be of a character and used in an amount that will form a stable nanoemulsion, as described above. Typically, the surfactants chosen are also inexpensive and easy to obtain and work with.

Specific examples of surfactants include polyoxyethylene alkyl ethers, such as laureth-2, -3, -4, -5, -7, -9, -10, -12, -15, -20, -23, -50, ceteth-2, -3, -5, -7, -12, -13, -15, -20, -50, oleth-2, -3, -4, -5, -7, -8, -10, -12, -20, -30, -40, -50, -70, ceteareth-3, -7, -10, -15, -20, -30; trideceth-3, -4, -5, -6, -7, -8, -10, -14, octyldodeceth-5, -8, PEG-7 cocoate, PEG-9 cocoate, PEG-2 oleate, PEG-3 oleate, PEG-5 oleate, PEG-6 oleate, PEG-7 oleate, PEG-8 oleate, PEG-9 oleate, PEG-10 oleate, PEG-14 oleate, PEG-2.5 castor oil, PEG-3 castor oil, PEG-12 castor oil, PEG-13 castor oil, PEG-17 castor oil, PEG-25 castor oil, PEG-32 castor oil, PEG-35 castor oil PEG-40 castor oil, PEG-54 castor oil, PEG-10 hydrogenated castor oil, PEG-12 hydrogenated castor oil, PEG-16 hydrogenated castor oil, PEG-20 hydrogenated castor oil, PEG-30 hydrogenated castor oil, PEG-60 hydrogenated castor oil, or PEG-100 hydrogenated castor oil; glyceryl fatty esters, such as glyceryl stearate, polyglyceryl-2 laurate, polyglyceryl-2 stearate, polyglyceryl-2 oleate, polyglyceryl-10 laurate, polyglyceryl-10 stearate, or polyglyceryl-10 oleate; sorbitan fatty esters and derivatives, such as sorbitan laurate, sorbitan palmitate, sorbitan stearate, sorbitan oleate, sorbitan trioleate, polysorbate 20, polysorbate 60, polysorbate 80, or polysorbate 85; alkanol amide surfactants, such as cocamide MEA or cocamide DEA; polyoxyethyleneoxypropylene glycol copolymers, such as poloxamer 61, 62, 64, 68, or 74; polyethylene glycol and derivatives, such as PEG-200, -300, -400, -600, -800, -1000, -1500, -2000, -3000, -4000, -4000F, -6000F, -8000, -15000, PEG-1000 stearate, PEG-1500 stearate, PEG-6000 stearate, PEG-400 distearate, PEG-6000 distearate, PEG-8000 distearate, PEG-300 oleate, PEG-400 oleate, PEG-6000 oleate, PEG-200 dioleate, or PEG-400 dioleate.

It will be understood that the surfactants described herein can be in a dry form or in a liquid form. When in dry form, a “solvent” is used in which the surfactant(s) is typically soluble. Examples of suitable solvents include water, hydrocarbons, and mixtures thereof. The solvent is typically water, so that the resulting composition is non-flammable and safe to transport.

“Non-turbid” means a solution that is substantially clear or transparent to the naked eye and that may be comparable to, for example, deionized water. For example, surfactants, such as two different polyethoxylated surfactants, are combined in amounts such that a non-turbid solution is formed. In some aspects, the nanoemulsion compositions have a color, sheen, or reflection, such as a light yellow or light blue.

“Substantially free” herein means less than about 5%, typically less than about 2%, more typically less than about 1%, even more typically less than about 0.5%, most typically less than about 0.1% contamination with the agent in question, such as a lower alkyl alcohol.

The term “lower alkyl alcohol” means a (C₁ to C₄) linear or branched alkyl alcohol, such as methanol, ethanol, propanol, butanol, isopropanol, 2-butanol, isobutanol, tert-butanol, ethylene glycol, propylene glycol, and glycerol.

The term “preservative” means a material that prevents the growth and or reacts with and/or destroys microorganisms that might damage or grow on or in the nanoemulsion composition or otherwise contaminate it.

Examples of preservatives include antimicrobial agents such as quaternary ammonium compounds, alcohols, chlorinated phenols, parabens and paraben salts, imidazolidinyl urea, phenoxyethanol, p-hydroxybenzoate, small carboxylic acids like benzoic acid, sorbic acid, salicylic acid, formic acid, proponic acid or corresponding salts. Formaldehyde-releasers and isothiazolinones may also be used. Typical non-limiting examples include DMDM hydantoin, diazolidinyl urea, imidazolidinyl urea, formaldehyde, propylparaben, ethylparaben, butylparaben, methylparaben, benzylparaben, isobutylparaben, phenoxyethanol, sorbic acid, benzoic acid, methylchloroisothiazolinone, methylisothiazolinone, methyl dibromoglutaronitrile, dehydroacetic acid, sodium bisulfite, dichlorophen, caprilyl glycol, salts of any of the foregoing compounds, and mixtures of any of the foregoing compounds. In typical aspects, the preservative comprises an isothiazolinone mixture, such as a mixture of methylchloroisothiazolinone and methylisothiazolinone, typically in a 3:1 ratio, as is included in the preservative Biocontrole™. In other typical aspects, the preservative comprises phenoxyethanol, as is included in the preservative Optiphen™.

The preservative, when present, is typically used in an amount of from about 0.01% to about 1% w/w, such as from about 0.01%, about 0.05%, about 0.1%, about 0.15%, about 0.2%, about 0.25%, about 0.5%, or about 0.75% to about 0.05%, about 0.1%, about 0.15%, about 0.2%, about 0.25%, about 0.5%, about 0.75%, or about 1% w/w. Typically, the preservative is used in an amount of about 0.15% w/w.

The term “chelator” or “chelating agent” means a molecule that can form coordinate bonds to a single metal atom—that is, it is a sequestering agent. Chelators bind to metal ions to form a metal/chelate complex and are used to mitigate changes in colour, texture, and/or fragrance in the compositions described herein. Chelating agents typically have two or more electron donating groups, and include, but are not limited to, ethylenediamine tetraacetic acid (EDTA), citric acid, tartaric acid, ascorbic acid, polyphosphates, esters or salts thereof, and combinations of any thereof. Typically, the chelator is EDTA.

The chelator, when present, is typically used in an amount of from about 0.01% to about 1% w/w, such as from about 0.01%, about 0.05%, about 0.1%, about 0.15%, about 0.2%, about 0.25%, about 0.5%, or about 0.75% to about 0.05%, about 0.1%, about 0.15%, about 0.2%, about 0.25%, about 0.5%, about 0.75%, or about 1% w/w. Typically, the chelator is used in an amount of about 0.15% w/w. In aspects, a chelator is absent from the compositions described herein.

The term “antioxidant” is used herein to define compounds that typically inhibit oxidation of an oxidation susceptible compound by reacting preferentially with the oxidizing agent before the oxidizing agent reacts with the compound. The product of the reaction between the antioxidant and the oxidizing agent is typically inert or non-reactive and tasteless, colorless, and odorless so as not to affect the taste, smell, or look of the product. Water soluble antioxidants suitable for use in the nanoemulsion compositions described herein include, but are not limited to, ascorbic acid, erythorbic acid, a botanical extract, such as rosemary extract, green tea extract, or other extract containing a polyphenol antioxidant, and combinations thereof. Oil soluble antioxidants suitable for use in the nanoemulsion compositions described herein include, but are not limited to, vitamin E, tocopherols, ascorbyl palmitate, butylated hydroxyanixole (BHA), butylated hydroxytoluene (BHT), pentaerythrityl tetra-di-t-butyl hydroxyhydrocinnamate, and combinations of any thereof. Typically, the antioxidant is BHT or pentaerythrityl tetra-di-t-butyl hydroxyhydrocinnamate, commercially available as Tinogard TT™.

The antioxidant, when present, is typically used in an amount of from about 0.01% to about 1% w/w, such as from about 0.01%, about 0.05%, about 0.1%, about 0.15%, about 0.2%, about 0.25%, about 0.5%, or about 0.75% to about 0.05%, about 0.1%, about 0.15%, about 0.2%, about 0.25%, about 0.5%, about 0.75%, or about 1% w/w. Typically, the antioxidant is used in an amount of about 0.10% w/w.

The term “cosmetically acceptable and/or pharmaceutically-acceptable” means that perfumes, additives, excipients, and/or inert ingredients modified by the term are suitable for use in contact with the tissues of humans and lower animals without undue toxicity, incompatibility, instability, irritation, allergic response, and the like, commensurate with a reasonable benefit/risk ratio.

The term “non-toxic” refers to the non-occurrence of pathological phenomena as a result of using the nanoemulsion compositions described herein. The term substantially non-toxic is defined as including acceptably low toxicity as well as non-toxicity.

The term “cloud point” indicates a temperature at which the nanoemulsion composition begins to scatter light, such that the surface of the nanoemulsion composition appears cloudy or opaque.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in aspects, the use of the oleth and/or steareth family of surfactants, or more specifically oleth-20, steareth-20, and/or steareth-21 is explicitly excluded from the compositions and methods described herein. In further aspects, the use of glycols is explicitly excluded from the compositions and methods described herein. In further aspects the use of an ionic surfactant, such as a cationic and/or anionic surfactant is explicitly excluded from the compositions and methods described herein. In further aspects, the use of ester surfactants and/or ester ether surfactants, such as pegylated fatty acids, is explicitly excluded from the compositions and methods described herein. In further aspects, the use of fatty acids is explicitly excluded from the compositions and methods described herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Methods of Producing Nanoemulsion Compositions

The nanoemulsion compositions described herein can be made by any known method. In one aspect, the nanoemulsion compositions are made by mixing one or more surfactants with a fragrance and an optional antioxidant and heating. When the temperature reaches about 70° C., a portion of an aqueous medium, such as water, is added. The composition is further mixed and heat to about 100° C. This temperature is maintained for a period of time, after which the remaining portion of the aqueous medium, such as water, is added, and the composition is quickly cooled. Any further desired excipients or additives, such as preservatives can be added at that time, with stirring or mixing to enhance solubilization. This method tends to result in a nanoemulsion with a smaller diameter size, such as less than about 100 nm.

In another aspect, the nanoemulsions are made by mixing the oil phase components and heating to from about 85° C. to about 95° C., mixing the aqueous phase components and heating to from about 85° C. to about 95° C., then adding the aqueous phase to the oil phase. The mixture is stirred for about 15 minutes and is then quickly cooled to about ambient temperature. Any further desired excipients or additives, such as preservatives can be added at that time, with stirring or mixing to enhance solubilization. This method tends to result in a nanoemulsion with a larger diameter size, such as from about 100 to about 300 nm.

In an aspect, the nanoemulsion compositions are prepared in a reactor in a water bath with a mechanical stirring mechanism. The one or more surfactants, fragrance and/or oil, and optional antioxidant are heated in the reactor with stirring to a temperature of from about 40° C. to about 80° C., such as from about 40° C., about 45° C., about 50° C., about 55° C., about 60° C., about 65° C., about 70° C., or about 75° C., to about 45° C., about 50° C., about 55° C., about 60° C., about 65° C., about 70° C., about 75° C., or about 80° C. In typical aspects, the one or more surfactants, fragrance, and optional antioxidant are heated to a temperature of about 70° C. In other typical aspects, the one or more surfactants, oil, and optional antioxidant are heated to a temperature of about 85° C. to about 95° C.

The portion of the aqueous medium that is added to the mixture of the one or more surfactants, fragrance and/or oil, and optional antioxidant is from about 20% to about 80% of the total amount to be added, such as from about 20%, about 25%, about 30%, about 35%, about 40%, about 45%, about 50%, about 55%, about 60%, about 65%, about 70%, or about 75% to about 25%, about 30%, about 35%, about 40%, about 45%, about 50%, about 55%, about 60%, about 65%, about 70%, about 75%, or about 80% of the total amount to be added. Typically, about 40% of the total amount of the aqueous medium is added.

Next, the mixture is further heated to the temperature at which the cloud point of the mixture is observed. In an aspect, this temperature is from about 85° C. to about 100° C., such as from about 85° C., about 86° C., about 87° C., about 88° C., about 89° C., about 90° C., about 91° C., about 92° C., about 93° C., about 94° C., about 95° C., about 96° C., about 97° C., about 98° C., or about 99° C. to about 86° C., about 87° C., about 88° C., about 89° C., about 90° C., about 91° C., about 92° C., about 93° C., about 94° C., about 95° C., about 96° C., about 97° C., about 98° C., about 99° C., or about 100° C. Typically, the cloud point is observed at about 95° C.

The cloud point temperature is maintained for a period of time, such as from about 1 to about 15 minutes, such as from about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, or about 14 minutes to about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes. In typical aspects, the cloud point temperature is maintained for about 5 minutes. In other typical aspects, the cloud point temperature is maintained for about 10 minutes. In other typical aspects, the cloud point temperature is maintained for about 15 minutes.

The composition is then quickly cooled, typically with the aid of a cooling system adapted to the reactor. Additionally, the remaining portion of the aqueous medium, at room temperature, is added. In an aspect, the composition is cooled to a desired temperature of from about 25° C. to about 60° C., such as from about 25° C., about 30° C., about 35° C., about 40° C., about 45° C., about 50° C., or about 55° C. to about 30° C., about 35° C., about 40° C., about 45° C., about 50° C., about 55° C., or about 60° C. Typically, the composition is cooled to a temperature of about 40° C.

Once the composition is cooled to the desired temperature, such as about 40° C., any further excipients and/or additives are added, such as a preservative. The further excipients and/or additives are typically added with stirring so that they are completely solubilized in the composition.

Nanoemulsion compositions produced by the methods described herein are, in aspects, non-toxic, suitable for use on human bodies, and/or non-flammable.

### Uses of Nanoemulsion Compositions

The nanoemulsion compositions described herein can be used for any known purpose, such as in air fresheners, perfumes, wax products (such as candles and melted wax pots), insect repellents, cosmetic products such as moisturizers, lotions, creams, soaps, shampoos, conditioners, and so forth. The nanoemulsion compositions described herein could be incorporated into other products to provide a scented object. In the particular case of insect repellants and cosmetic products, the nanoemulsion compositions may be suitable for application to a living subject. In certain aspects, the compositions described herein are suitable as a raw material for formulating many different products. In certain aspects, the nanoemulsion compositions described herein are substantially odorless and therefore particularly suitable for application to skin as, for example, a moisturizer, or as a base product for further formulation.

The above disclosure generally describes the present invention. A more complete understanding can be obtained by reference to the following specific Examples. The Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

In the following Examples 1-4, the nanoemulsions were prepared as follows. A 100 L reactor comprising a mechanical stirring mechanism and heating provided via an external water bath jacket was set to heat to 95° C.

The oleth-20, laureth-2 (when used), fragrance, and BHT were added to the reactor with stirring. When the reactor temperature reached 70° C., 40% of the total water volume was added, with stirring.

When the reactor reached about 95° C., the cloud point was observed, meaning an aspect of “cloud” was visible on the composition surface. This temperature was maintained for about 5 minutes and the composition was cooled quickly by adding the remaining 60% of the total water volume together with the aid of a cooling system adapted to the reactor. When the reactor temperature reached 40° C., the methylchloroisothiazolinone and methylisothiazolinone were added with stirring until they were completely solubilised.

As will be shown, the resultant nanoemulsion had a droplet size in the range of from about 15 nm to about 30 nm, considering intensity distribution, and the fragrance was more stable in nanoemulsion form than it was when simply combined with a vehicle.

In the following Examples 5-7, the nanoemulsions were prepared as follows. A 100 L reactor comprising a mechanical stirring mechanism and heating provided via an external water bath jacket was set to heat to 85-95° C.

The laureth-4 and laureth-23 or oleth-20 and laureth-4, oil, and Tinogard TT were added to the reactor with stirring. The water was separately heated to the same temperature and was added to the heated oil components. The resulting composition was stirred for about 15 minutes and was quickly cooled to ambient temperature. The Optiphen was added with stirring until it was completely solubilised.

As will be shown, the resultant nanoemulsion had a droplet size in the range of from about 100 nm to about 350 nm, considering intensity distribution.

### Example 1

A nanoemulsion containing the following components was prepared:

| Component | Quantity (% w/w) |
|---|---|
| Oleth-20 | 10.8 |
| Laureth-2 | 1.2 |
| Fragrance | 10 |
| BHT | 0.10 |
| Biocontrole | 0.10 |
| Water | qsp 100 |

### Example 2

A nanoemulsion containing the following components was prepared:

| Component | Quantity (% w/w) |
|---|---|
| Oleth-20 | 14.4 |
| Laureth-2 | 1.6 |
| Fragrance | 10 |
| BHT | 0.10 |
| Biocontrole | 0.10 |
| Water | qsp 100 |

### Example 3

A nanoemulsion containing the following components was prepared:

| Component | Quantity (% w/w) |
|---|---|
| Oleth-20 | 10.0 |
| Fragrance | 5-10 |
| BHT | 0.10 |
| Biocontrole | 0.10 |
| Water | qsp 100 |

### Example 4

A nanoemulsion containing the following components was prepared:

| Component | Quantity (% w/w) |
|---|---|
| Oleth-20 | 5.40 |
| Laureth-2 | 0.60 |
| Citronella essential oil | 2 |
| BHT | 0.10 |
| Biocontrole | 0.10 |
| Water | qsp 100 |

### Example 5

A nanoemulsion containing the following components was prepared:

| Component | Quantity (% w/w) |
|---|---|
| Oleth-20 | 2.7 |
| Laureth-4 | 0.30 |
| Citronella essential oil | 10 |
| Tinogard TT | 0.10 |
| Optiphen | 1.0 |
| Water | qsp 100 |

### Example 6

A nanoemulsion containing the following components was prepared:

| Component | Quantity (% w/w) |
|---|---|
| Laureth-23 | 2.4 |
| Laureth-4 | 3.6 |
| Carapa guianensis oil | 5 |
| Tinogard TT | 0.10 |
| Optiphen | 1.0 |
| Water | qsp 100 |

### Example 7

A nanoemulsion containing the following components was prepared:

| Component | Quantity (% w/w) |
|---|---|
| Oleth-20 | 2.4 |
| Laureth-4 | 0.6 |
| Lemon grass essential oil | 7.5 |
| Tinogard TT | 0.10 |
| Optiphen | 1.0 |
| Water | qsp 100 |

### Example 8

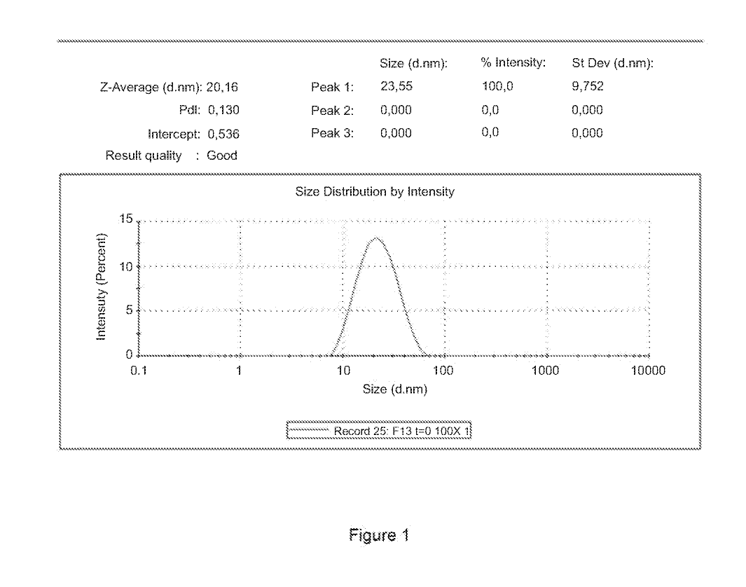

The nanoemulsion of Example 1 was diluted 100 times with water and the average droplet size (Zeta-average; d nm) and polydispersity (Pdl) was measured using a Zetasizer ZEN3600 (Malvern). FIG. 1 shows that the nanoemulsion contains droplets in the nanometer size.

### Example 9

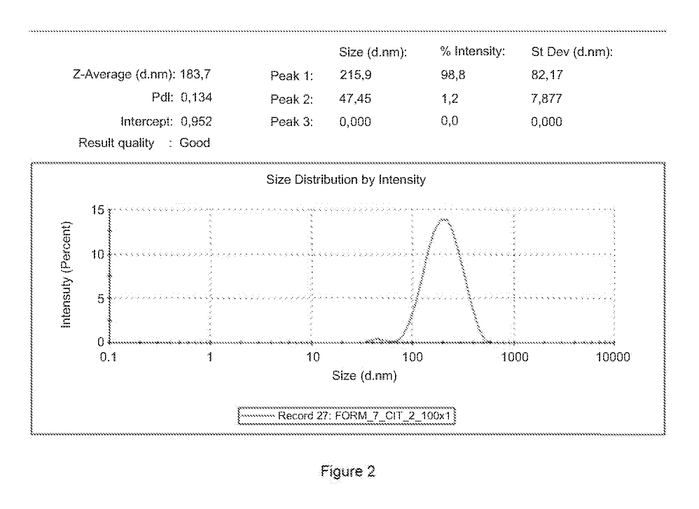

The nanoemulsion of Example 5 was diluted 100 times with water and the average droplet size (Zeta-average; d nm) and polydispersity (Pdl) was measured using a Zetasizer ZEN3600 (Malvern). FIG. 2 shows that the nanoemulsion contains droplets in the nanometer size.

### Example 10

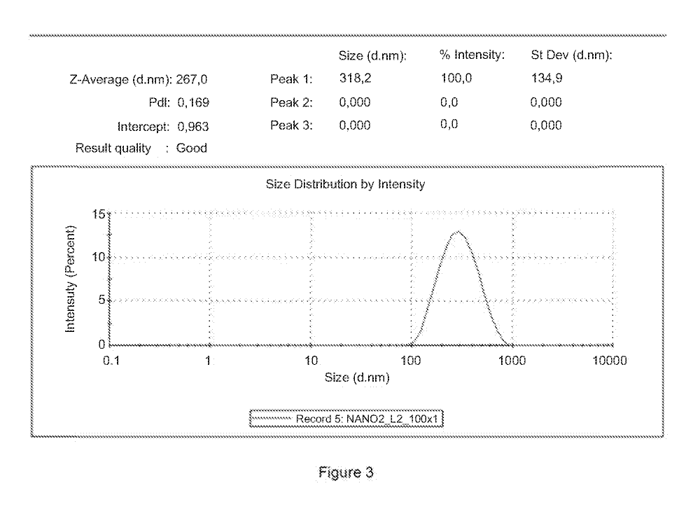

The nanoemulsion of Example 6 was diluted 100 times with water and the average droplet size (Zeta-average; d nm) and polydispersity (Pdl) was measured using a Zetasizer ZEN3600 (Malvern). FIG. 3 shows that the nanoemulsion contains droplets in the nanometer size.

### Example 11

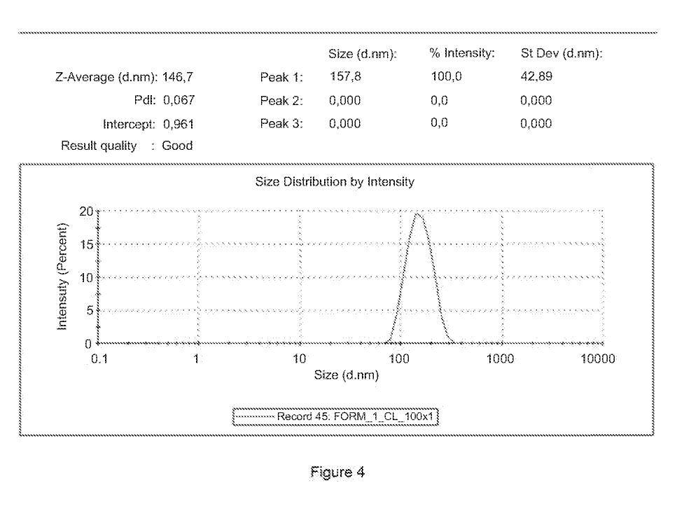

The nanoemulsion of Example 7 was diluted 100 times with water and the average droplet size (Zeta-average; d nm) and polydispersity (Pdl) was measured using a Zetasizer ZEN3600 (Malvern). FIG. 4 shows that the nanoemulsion contains droplets in the nanometer size.

### Example 12

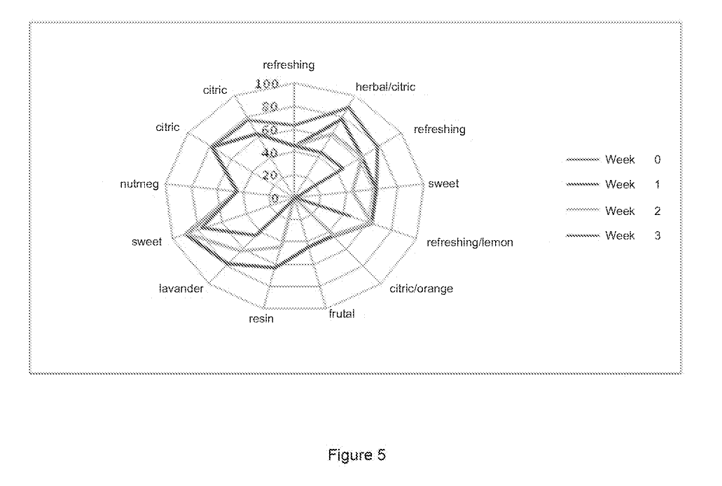

FIG. 5 shows the olfactory characteristics of a nanoemulsion composition comprising an essential oil and a vehicle, as measured using an olfactometer. The olfactometer uses gas chromatography analysis to quantify specific smells together with a trained technique to identify them.

### Example 13

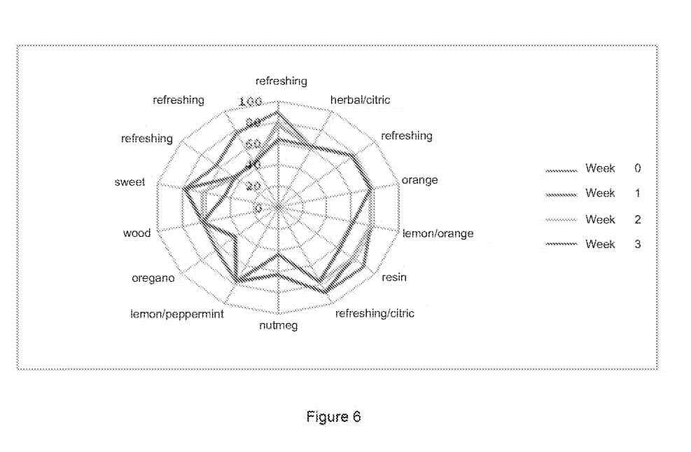

FIG. 6 shows the olfactory characteristics of a nanoemulsion composition comprising an essential oil in a nanoemulsion, as measured using an olfactometer. The olfactometer uses gas chromatography analysis to quantify specific smells together with a trained technique to identify them.

1. A composition comprising a fragrance and/or oil, a surfactant, and an aqueous medium, wherein the composition is free of lower alkyl alcohols and wherein the composition is a nanoemulsion.

2.-3. (canceled)

4. The composition of claim 1, wherein the surfactant comprises at least a first non-ionic ethoxylated fatty alcohol of the formula:
CH₃CH₂ₘO—CH₂—CH₂ₙOH;
wherein m is from about 4 to about 40 and n is from about 1 to about 60.

5.-6. (canceled)

7. The composition of claim 4, wherein the composition comprises a second ethoxylated fatty alcohol of the formula:
CH₃CH₂ₘO—CH₂—CH₂ₙOH;
wherein m is from about 4 to about 40 and n is from about 1 to about 60, wherein the second ethoxylated alcohol is different from the first ethoxylated alcohol.

8. The composition of claim 7, wherein m and n in the first ethoxylated alcohol are from about 12 to about 20 and from about 1 to about 10, respectively, and wherein m and n in the second ethoxylated alcohol are from about 12 to about 20 and from about 11 to about 30, respectively.

9. The composition of claim 1, wherein the surfactant comprises oleth-20, laureth-2, laureth-4, laureth-23, or a combination thereof.

10. The composition of claim 9, wherein the surfactant consists of oleth-20 and laureth-2, oleth-20 and laureth-4, laureth-23 and laureth-4, or laureth-23 and laureth-2.

11. The composition of claim 10, wherein the surfactant consists of oleth-20.

12. (canceled)

13. The composition of claim 1, wherein the surfactant is present in the composition in a total amount of from about 5% w/w to about 18% w/w.

14. (canceled)

15. The composition of claim 1, wherein the fragrance and/or oil is present at about 10%, about 5%, or about 2%.

16. (canceled)

17. The composition of claim 1, wherein the fragrance comprises an essential oil.

18. The composition of claim 17 wherein the essential oil is citronella oil or lemongrass oil.

19. The composition of claim 1, wherein the oil is andiroba oil.

20. The composition of claim 1, further comprising a preservative selected from antimicrobial agents, small carboxylic acids and salts thereof, formaldehyde-releasers, isothiazolinones, phenoxyethanol, caprilyl glycol, and combinations thereof.

21. (canceled)

22. The composition of claim 20, wherein the preservative is an isothiazolinone or a mixture of phenoxyethanol and caprilyl glycol.

23. The composition of claim 20, wherein the preservative is present in an amount of from about 0.01% to about 1% w/w.

24. The composition of claim 1, further comprising an antioxidant selected from ascorbic acid, erythorbic acid, a botanical extract, rosemary extract, green tea extract, an extract containing a polyphenol antioxidant, vitamin E, tocopherols, ascorbyl palmitate, butylated hydroxyanixole (BHA), butylated hydroxytoluene (BHT), pentaerythrityl tetra-di-t-butyl hydroxyhydrocinnamate, and combinations of any thereof.

25. (canceled)

26. The composition of claim 24, wherein the antioxidant is BHT or pentaerythrityl tetra-di-t-butyl hydroxyhydrocinnamate.

27. (canceled)

28. The composition of claim 1, wherein the nanoemulsion has an average droplet size of from about 1 nm to about 100 nm, from about 20 nm to about 50 nm, or from about 10 nm to about 30 nm.

29. The composition of claim 1, wherein the aqueous medium is water.

30. The composition of claim 1, wherein the composition is non-turbid, stable, free of an oleth-based surfactant, and/or free of fatty acids.

31.-32. (canceled)

33. The composition of claim 1, wherein the composition is free of one of more of oleth-20, a steareth-based surfactant, steareth-20, steareth-21, glycols, an ionic surfactant, ester surfactants, ester ether surfactants, and pegylated fatty acids.

34.-41. (canceled)

42. A method of making a composition, the method comprising:
heating a mixture of a fragrance and/or oil and a surfactant until it reaches a cloud point temperature; and
adding an aqueous medium to rapidly cool the mixture,
wherein the composition is a nanoemulsion.

43. The method of claim 42, wherein the aqueous medium is added in a first portion and a second portion and wherein heating the mixture of the fragrance and/or oil and the surfactant until it reaches a cloud point comprises:
heating the mixture of the fragrance and/or oil and the surfactant to a first temperature;
adding the first portion of the aqueous medium to the mixture; and
heating the mixture until it reaches the cloud point temperature.

44. (canceled)

45. The method of claim 43, wherein the first portion of the aqueous medium is about 40% of the total amount of the aqueous medium, wherein the first temperature is about 70° C., the cloud point temperature is about 95° C., the cloud point temperature is maintained for about 5 minutes, the composition is cooled to a temperature of about 40° C., and/or the components are stirred during heating and/or cooling.

46.-95. (canceled)

96. A room diffuser, perfume, air freshener, insect repellent, or cosmetic product comprising the composition of claim 1.

97.-102. (canceled)
